(19) United States
(12) Patent Application Publication
(10) Pub. No.: US 2020/0298271 A1
(43) Pub. Date: Sep. 24, 2020

(54) DRY POWDER COATING APPARATUS

(71) Applicant: Ningbo Weston Powder Pharma Coatings Co., Ltd., Ningbo (CN)

(72) Inventors: JIE DONG, NINGBO (CN); JINGXU ZHU, LONDON (CA); QINGLIANG YANG, Ningbo (CN); YINGLIANG MA, LONDON (CA); KAIQI SHI, Ningbo (CN); HANG ZHOU, Ningbo (CN); SHOUYUN ZHOU, Ningbo (CN)

(21) Appl. No.: 16/519,294

(22) Filed: Jul. 23, 2019

(30) Foreign Application Priority Data
Jul. 27, 2018 (CN) 201810851086.7

(51) Int. Cl. B05D 1/06; B05B 5/08

(52) U.S. Cl. CPC B05D 1/06; A61K 9/2893; B05B 5/087

(57) ABSTRACT

A dry powder coating apparatus for coating pharmaceutical solid dosage forms includes a housing; a drum coater; a powder coating system; a liquid spray system; a ventilation system; a heating system; a touch screen control panel; and an operation box. The drum coater may have a truncated cone on both sides, in which one side is opened and another side is closed. The drum coater is horizontally placed inside the operation box along its axis and fixed on a rotatable shaft and a motor drives the rotatable shaft and rotates the drum coater about its own axis.

### FIELD

This disclosure provides an apparatus which is used for dry powder coating of pharmaceutical solid dosage forms.

### BACKGROUND

Pharmaceutical solid dosage forms are normally coated with one or more layers of thin film which can mask the bad odor emitted by drugs and/or other excipients, prevent certain substances in the drug forms being unstable or oxidized in the air, control drugs' release site and release rate, reduce the breakage rate of drug cores in high-speed packaging equipment and improve drug identification, etc.

Generally, the coating methods of the solid dosage forms, depending on the techniques used, can be divided into two categories: wet coating methods and dry coating methods. At present, coating apparatus based on the wet coating methods are widely used in pharmaceutical industry to coat solid dosage forms.

The working principle of the wet coating apparatus is to dissolve/disperse the coating materials into organic solvents/water to form solutions/suspensions, then the solutions/suspensions are sprayed on the surface of solid dosage forms loaded inside a rotating drum coater. After that, hot air blows towards the surface of wetted solid dosage forms to accelerate the evaporation of the solutions/suspensions, and finally a protective polymer film is uniformly formed on the surface of solid dosage forms. Although, the organic solvents based wet coating can make the film formation faster and more uniform due to the dissolved nature of coating polymers, the toxic organic solvents reduce the safety of the drugs and cause environmental problems.

As a result, aqueous coating, where water is used as the solvent, started to dominate in 1990s and remains the preferred coating approach in the pharmaceutical industry. Nevertheless, aqueous coating still has many limitations, such as long processing time and high energy consumption. In addition, aqueous coating is not appropriate for coating the drugs which contain moisture sensitive active pharmaceutical ingredients.

The dry coating technologies have been continuously developed and improved in recent years to overcome above problems of wet coating methods. Compared with the wet coating methods, the dry coating methods could directly form the coating film on the surface of solid dosage forms without adding any organic solvent or water. Hence, the dry coating methods greatly reduce coating time, save energy, and effectively avoid environmental pollutions caused by the discharge of organic solvents.

At present, dry coating methods mainly include electrostatic dry powder coating, plasticizer-dry powder coating and plasticizer-electrostatic dry powder coating. Since the last method combines the former two ones, integrates their advantages and overcomes their defects, it has high potential of research and market applications. However, in both domestic and international markets, the coating apparatus based on the wet coating methods are still widely used for coating the solid dosage forms in pharmaceutical industry. The dry powder coating apparatus have been only theoretically studied in lab scale due to their high manufacturing costs and complex internal structures. Reports on industrial dry powder coating apparatus are still very rare.

The deficiencies of current lab scale dry powder coating apparatus include the following.

a) The dry powder coating apparatus normally heat and dry solid dosage forms by means of electric heating wires or infrared ray which has low thermal efficiency and consumes plenty of energy.
b) Operators have to manually operate a corona spray gun and insert the nozzle of the spray gun into a drum coater to spray the charged coating powder to the surface of drug cores. Since a high-voltage electrode is installed inside a corona spray gun and the operators need to observe the coating process at a close distance, operators may have risk of suffering an electric shock.
c) It is difficult for operators to clearly observe and aim the gun nozzle at the moving bed of the drug cores when the drum coater is full of charged powder, which may waste the coating materials.
d) For some dry powder coating apparatus, the coating powder is electrostatically pretreated by an external fluidized bed and the charged coating powder is then piped into a drum coater to reduce the risk of electric shock to operators. However, the industrial fluidized beds are quite expensive, take up a lot of working space, and need regular maintenance. Besides, it takes much time to thoroughly clean the fluidized beds and the powder conveying pipelines when a new material will be served as the coating powder.
e) The wall of drum coater is normally perforated, which wastes a lot of coating materials and cannot be used for coating the pellets.
f) The coating apparatus have poor internal ventilation. There will be a risk of dust explosion, if a high concentration of dry powder meets a high voltage static electricity which is generated by an electrostatic spray gun.
g) The drum coaters are usually placed obliquely. The inclined cylindrical drum coaters may reduce the surface area of drug cores under the spray zone and shorten the residence time of drug cores under the spray region. As a result, the inclined drum coaters may reduce the powder coating efficiency, prolong the coating time, waste energy, lower the uniformity of coating layer, etc.
h) The drum coaters are made of stainless-steel. Since the electrical resistance of drug pills is much larger than that of stainless-steel, a majority of charged coating powder prone to adhere to the inner surface of the drum coaters during the process of powder coating. The stainless-steel drum coaters not only waste coating materials, but are also difficult to clean.

In view of the above defects of current lab scale coating apparatus, it is necessary to design a dry powder coating apparatus which has simple structure, high coating efficiency, low energy consumption, is safe to operate, and meets the needs for coating both pellets and ordinary tablets in industrial production.

### SUMMARY

The present disclosure provides a dry powder coating apparatus for coating pharmaceutical solid dosage forms comprising a housing, a drum coater, a powder coating system, a liquid spray system, a heating system, a ventilation system and a touch screen control panel.

In the process of dry powder coating, the heating system works and heats the drug cores inside the drum coater which is rotating around its own axis. Meanwhile, the ventilation system works continuously to reduce powder accumulation inside the drum coater. The powder coating system and liquid spray system spray coating powder and auxiliary materials (e.g. plasticizer), respectively, to the surface of the drug cores loaded inside the rotating drum coater.

The powder coating system and liquid spray system can synchronously or asynchronously perform multiple spraying operations on the drug cores. Operators can observe and adjust the operation conditions of the drum coater through the touch screen control panel which is fixed on the housing. As the drum coater keeps rotating, the coating powder uniformly adhere to the surface of drug cores. Through continuous heating, which promotes the curing of polymers contained in the coating powder, a uniform film is formed on the surface of solid dosage forms.

The dry powder coating apparatus disclosed here is easy to maintain and to operate. It greatly shortens the coating time, is energy-saving and environmentally friendly.

Various exemplary embodiments of the present disclosure provide a dry powder coating apparatus for coating pharmaceutical solid dosage forms.

Thus there is provided a dry powder coating apparatus for coating pharmaceutical solid dosage forms, comprising:

a drum coater having a hollow interior, the drum coater being mounted inside a housing and being rotatable about its longitudinal axis;

a powder coating system for spraying a dry powder inside the hollow interior of the drum coater;

a liquid spray system for spraying a liquid containing auxiliary materials inside the hollow interior of the drum coater;

a heating system for heating the hollow interior of the drum coater, the heating system being positioned adjacent to the drum coater;

an operation box that encloses the drum coater in an operation volume and the operation box separates the operation volume from an external environment;

a ventilation system for air circulation between the operation volume and the external environment, wherein the ventilation system has an air inlet duct and an air outlet duct; and a touch screen control panel attached to the housing and the touch screen control panel is configured to display at least one operating parameter of the dry powder coating apparatus.

The drum coater of the dry powder coating apparatus may have a truncated cone on both sides, in which one side is opened and another side is closed.

The drum coater may be horizontally placed inside the operation box along its axis and fixed on a rotatable shaft.

A motor may be used to drive the shaft of the drum coater and rotates the drum coater about its longitudinal axis.

The drum coater may be horizontally placed inside the operation box along its longitudinal axis and fixed on a rotatable shaft.

A motor may be mechanically connected to the drum coater such that the motor can rotate the drum coater about the longitudinal axis.

The wall of drum coater may be non-perforated, fully perforated or partially perforated. In an embodiment, each hole size in the fully or partially perforated wall may be around 1-10 mm, depending on the size of tablets to be coated.

The dry powder coating apparatus may further comprise at least two or more baffles installed inside the wall of the drum coater. The baffles and the inner wall of the drum coater may be fully or partially coated with or made of insulating materials.

In an embodiment, the powder coating system is for forming a charged dry powder spray zone inside the drum coater, which comprises at least one corona spray gun which is fixed on at least one rotatable telescopic support, a powder container, powder delivery conduits which connect the powder container and a high-pressure gas source to the corona spray gun.

In an embodiment, the liquid spray system is for forming a liquid spray zone inside the drum coater, which comprises at least one atomizer which is fixed on at least one rotatable telescopic support, a metering pump, a liquid container, liquid delivery conduits which connect the atomizer with the metering pump and the liquid container, and gas delivery conduits which connect the atomizer with a high-pressure gas source.

The corona spray gun may have a nozzle having a variety of 0-90° spray angle options for controlling the direction and the shape of the corona spray gun's spray zone. The corona spray gun may share a rotatable telescopic support with the atomizer.

The liquid spray zone may be above the charged dry powder spray zone.

One side of the rotatable telescopic support may be fixed on the housing, and another side may be inserted into the drum coater and adjusted above drug cores by touching the control buttons on the touch screen control panel.

The ventilation system may include a trapezoidal duct above the drum coater.

The heating system may include heating wires inside the air inlet duct and an electromagnetic heater which is installed below the drum coater.

The drum coater may be heated by a power adjustable electromagnetic heater installed below the coater or by the electric heating wires inside the air inlet duct, or simultaneously heated by both power adjustable electric heating wires and electromagnetic heater.

The touch screen control panel may be fixed on the housing. The touch screen control panel displays the operation conditions of the drum coater selected from the group consisting of temperature, humidity, rotation speed, the amount of powder sprayed, flow rate, location of rotatable telescopic support, and systems errors.

The operation box may be inside the housing. The drum coater may be cylindrically shaped.

The air outlet duct may have a filter therein and being connected to a fan such that the ventilation system is configured to generate negative air pressure in the operation volume.

Further features will be described or will become apparent in the course of the following detailed description.

### BRIEF DESCRIPTION OF THE DRAWINGS

Embodiments will now be described, by way of example only, with reference to the accompanying drawings, in which:

FIG. 1 is a front view of the dry powder coating apparatus.

FIG. 2 is a side view of the dry powder coating apparatus.

FIG. 3 is a side view of the non-perforated drum coater.

FIG. 4 is a side view of the partially-perforated drum coater.

FIG. 5 is a side view of the fully-perforated drum coater.

### DETAILED DESCRIPTION OF THE EMBODIMENTS

Various embodiments and aspects of the disclosure will be described with reference to details discussed below. The following description and drawings are illustrative of the disclosure and are not to be construed as limiting the disclosure. The drawings are not necessarily to scale. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

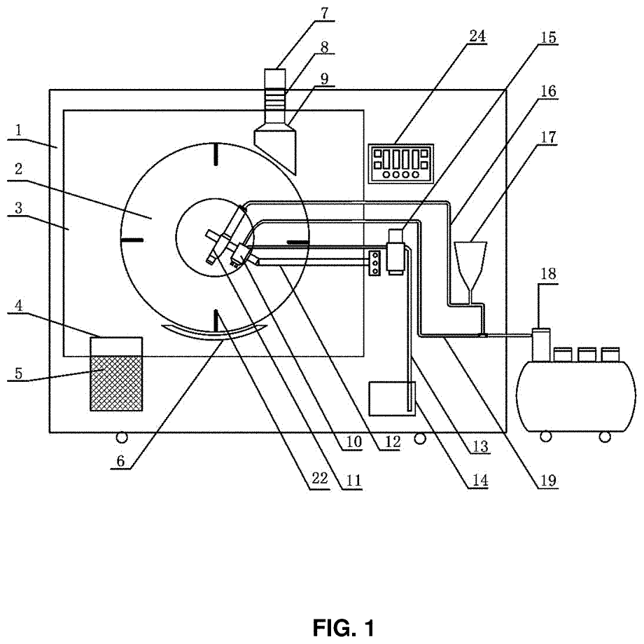

Embodiments of the dry powder coating apparatus comprise of the following components in reference to the FIGS. 1 to 5.

### PARTS LIST

1: Housing
2: Drum coater
3: Operation box
4: Air outlet
5: Filter
6: Electromagnetic heater
7: Air inlet duct
8: Electric heating wires
9: Trapezoidal duct
10: Atomizer
11: Corona spray gun
12: Rotatable telescopic support
13: Liquid delivery conduit
14: Liquid container
15: Metering pump
16: Powder delivery conduit
17: Powder container
18: High-pressure gas source
19: Gas delivery conduit
20: Motor
21: Fan
22: Baffle
23: Dustproof door
24: Touch screen control panel

Under the background techniques described above, this disclosure provides a dry powder coating apparatus for coating pharmaceutical solid dosage forms. This apparatus has several advantages over the current lab scale dry powder coating apparatus, such as simple structure, safe to operate, high coating efficiency, low energy consumption, high efficiency coating of pellets or ordinary tablets, etc., which is ideal for industrial powder coating of pharmaceutical solid dosage forms.

The present disclosure relates to a dry powder coating apparatus for coating pharmaceutical solid dosage forms, comprising a housing, a drum coater, a powder coating system, a liquid spray system, a heating system, a ventilation system and a touch screen control panel. An operation box may be provided inside the housing of coating apparatus. The drum coater is mounted inside the operation box. The wall of drum coater is fully perforated, partially-perforated or non-perforated and suitable for coating the pellets or ordinary tablets. The powder coating system includes at least one corona spray gun fixed on one or more rotatable telescopic supports, a powder container, powder delivery conduits which connect the powder container and a high-pressure gas source to the corona spray gun.

The nozzle of the corona spray gun has a variety of 0-90° spray angle options for multi-angle spraying, thereby increasing the efficiency of powder spraying. The liquid spray system includes at least one atomizer fixed on one or more rotatable telescopic supports, a metering pump, a liquid container, liquid delivery conduits which connect the atomizer with the metering pump and the liquid container, and gas delivery conduits which connect the atomizer with a high-pressure gas source.

The ventilation system includes an air inlet duct installed at the top of the coating apparatus housing, a trapezoidal duct above the drum coater, an air outlet, a filter and a fan. The heating system includes heating wires inside the air inlet duct and an electromagnetic heater which is installed below the drum coater.

The drum coater has a truncated cone on both sides, in which one side is opened and another side is closed. The drum coater is horizontally placed inside the operation box along its axis and fixed on a rotatable shaft. A motor drives the shaft and rotates the drum coater about its own axis. These design features are beneficial for reducing the number of drug cores thrown out of the drum coater, when the drug cores are falling, flipping and rolling inside the coater.

One or more rotatable telescopic supports can be used in this disclosure. One end of the telescopic support is fixed to the housing. Another end extends into the interior of drum coater, which is rotatable and located above the drug cores inside the drum coater.

One or more corona spray guns can be used in this disclosure. The corona spray guns are fixed on one or more rotatable telescopic supports.

One or more atomizers can be used in this disclosure. The atomizers are fixed on one or more rotatable telescopic supports.

To achieve a higher powder coating efficiency, the spray zone of each atomizer is above the spray zone of each corona spray gun.

By adjusting one or more rotatable telescopic supports, the position of one or more corona spray guns and the position of one or more atomizers can be adjusted accordingly.

The nozzle of corona spray gun is replaceable, and it has a variety of 0-90° spray angle options, for multi-angle spraying and increasing the efficiency of dry powder coating.

The drum coater can be heated by a power adjustable electromagnetic heater installed below the coater or by power adjustable electric heating wires inside the air inlet duct. These two heating modules can work simultaneously or separately depending on different temperature requirements of powder coating process. The heating wires may also be replaced by another heating device such as an infrared heater. If necessary, one or more heating rollers can be installed around the drum coater.

The wall of drum coater is non-perforated, which can effectively coat pellets and ordinary tablets without spending extra money to purchase other coating apparatus such as fluidized beds. The wall of drum coater can be perforated or partial perforated with hole size of around 1-10 mm, which is suitable for coating the large drug cores.

There are at least two baffles installed inside the wall of drum coater. Each baffle is fixed at an angle of 0-90° to the axis of the drum coater.

An insulated dustproof door is provided on the operation box. By closing the door, an enclosed operation space is formed inside the operation box.

An air inlet duct, passing through the housing, is fixed above the operation box. An air outlet duct, a filter and a fan are installed below the operation box inside the housing. During the coating process, the fan pumps air from the air inlet duct towards the air outlet duct forming a negative pressure operation condition inside the operation box.

The touch screen control panel is configured to display the operation conditions of the drum coater such as temperature, humidity, rotation speed, the amount of powder sprayed by the corona spray gun, flow rate of the atomizer, location of the rotatable telescopic support, systems errors, etc. The above parameters can be set and adjusted by touching the buttons on the screen.

In the coating process, drug cores are loaded inside the rotatable drum coater which is driven by a motor and rotates around its own axis. The drug cores are heated by electric heating wires installed inside the air inlet duct or/and an electromagnetic heater fixed below the drum coater. Auxiliary coating materials such as plasticizer are kept in a liquid container, and they are sprayed to the surface of rotating drug cores loaded inside the drum coater through an atomizer to which the liquid delivery conduits, a metering pump and a liquid container connect.

The adding of plasticizer can reduce the surface resistance of drug cores and can enhance the adhesion rate of electrostatic powder to the surface of drug cores. The rotatable telescopic support can be adjusted by touching the buttons on the touch control panel. The nozzle of the corona spray gun and the atomizer fixed on the support can be adjusted and vertically aimed at the bed of rolling drug cores, which further improve the coating efficiency.

The nozzle of the corona spray gun has a variety of 0-90° spray angle options, which enables multi-angle spraying. The dry powder is kept in a powder container. The corona spray gun charges the dry powder and forms an electric potential difference between the charged powder and the drum coater. Under the effect of electric field force, the charged dry powder adheres to the surface of drug cores. The ventilation system forms a negative pressure working condition inside the operation box, which can effectively prevent accumulation of dust and odor. Through the constant rotating of drum coater, the auxiliary coating materials and dry powder uniformly adhere to the surface of drug cores. By heating the drug cores to a certain temperature such as the glass transition temperature of the polymers contained in the coating powder, a layer of uniform film is rapidly formed on the surface drug cores.

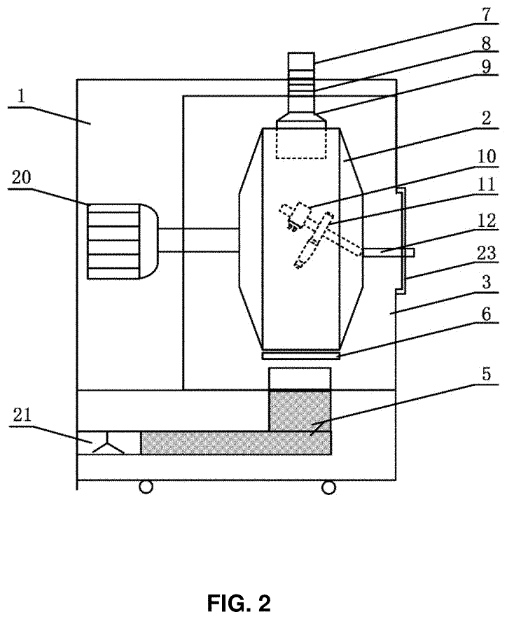

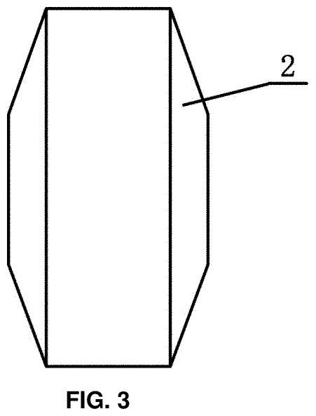

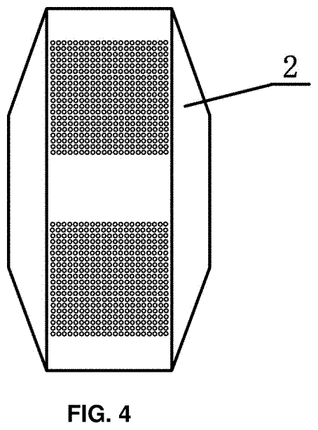

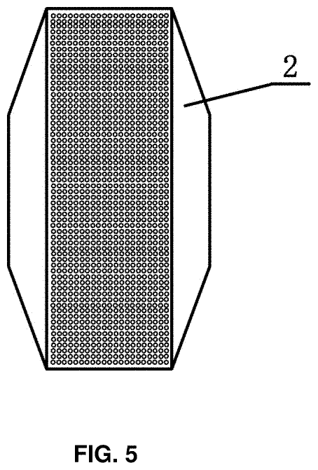

FIG. 1 and FIG. 2 shows front view and side view of the dry powder coating apparatus, respectively, according to an embodiment of the present disclosed. As shown in FIG. 1 and FIG. 2, the present embodiment comprises a housing 1, a drum coater 2, a powder coating system, a liquid spray system, a heating system, a ventilation system and a touch screen control panel 24. FIG. 3, FIG. 4 and FIG. 5 are side view of non-perforated drum coater, partially-perforated drum coater and fully-perforated drum coater respectively.

The housing 1 is provided with an operation box 3 with one side equipped with a dustproof door 23. The dustproof door 23 is made of transparent and electrical insulating materials such as glass or perspex, which shields the operators from being exposed to high-voltage electrostatic field and coating powder. The dustproof door 23 can also enhance the negative pressure working condition inside the operation box 3, hence effectively reducing the hazards to operators arising from dust inhalation.

The drum coater 2 is made of 304 stainless-steel, which has stable chemical properties, smooth internal structure and no dead angle and it follows the standard of Good Manufacturing Practice (GMP). The drum coater 2 is a truncated cone on both sides, in which one side is opened and another side is closed and fixed on a shaft of a motor 20. The drum coater 2 is horizontally placed inside the operation box 3 along its axis. The motor 20 drives the shaft and rotates the drum coater 2 about its own axis. The horizontally placed drum coater 2 not only maximizes the surface area of the drug cores exposed to the spray zones, but also prolongs the residence time of the drug cores on the surface of drug cores' bed. This horizontally placed drum coater 2 can improve the dry powder coating efficiency, shorten coating time, save energy, enhance the uniformity of coating film on drug cores, etc.

Baffles 22 are installed on the interior wall of drum coater 2, lifting, dropping and flipping the inside drug cores, which increase the contact time and contact area of the drug cores with the coating powder and auxiliary coating materials. Thereby, the installation of baffles 22 further improves the efficiency of dry powder coating. The number and position of baffles can be selected according to different filling levels of drug cores, the size and rotation speed of drum coater. Preferably at least two baffles can be used. The baffles are placed at an angle of 0-90° to the axis of the drum coater 2.

The wall of drum coater 2 can be non-perforated, enabling effective coating of pellets. In another embodiment, the wall of drum coater 2 can be perforated or partially perforated with hole size of about 1-10 mm for coating large drug cores. The baffles 22 and the inner surface of truncated cones of the drum coater are fully or partially coated with or made of insulating materials, which greatly reduce the amount of charged powder that adhere to the metal surface of the drum coater. Therefore, the fully or partially insulated drum coater not only saves coating materials, but also is easy to clean.

The insulating materials that are used to prevent charged powders from adhering to the inner surface of the stainless-steel drum coater may comprise, but not limited to, plastics, wood, glass, ceramic, rubber, mica, or any combination of above materials thereof, etc.

The powder coating system comprises a corona spray gun 11 which is fixed on a rotatable telescopic support 12, a powder container 17, powder delivery conduits 16 which connect the powder container 17 and a high-pressure gas source 18 to the corona spray gun 11. More than one corona spray guns 11 can be fixed on the rotatable telescopic support 12 depending on the size of drum coater 2 and filling level of drug cores.

The corona spray gun is made of food-grade plastics, following the standard of Good Manufacturing Practice (GMP), which may comprise, but not limited to, Poly tetra fluoroethylene (PTFE), Polyethylene terephthalate (PET), High Density Polyethylene (HDPE), Polyvinyl chloride (PVC), Low Density Polyethylene (LDPE), Polypropylene (PP), Polystyrene (PS), Polycarbonate (PC) and so on to give a few non-limiting examples.

One side of the rotatable telescopic support 12 is fixed to the housing 1. And another side can be inserted into the drum coater 2 and adjusted above the drug cores. This telescopic support 12 is rotatable and stretchable, so that the spray zones of the corona spray gun 11 and the atomizer 10 are adjustable by adjusting the location and direction of the telescopic support 12. By aiming the nozzle of corona spray gun 11 and atomizer 10 at the drug cores, the amount of dry coating powder and the auxiliary coating materials used in powder coating can be greatly reduced. More than one rotatable telescopic support 12 can be used depending on the size of drum coater.

The liquid spray system comprises one atomizer 10 fixed on the rotatable telescopic support 12, a metering pump 15, a liquid container 14, liquid delivery conduits 13 which connect the metering pump 15 and the liquid container 14 to the atomizer 10, and gas delivery conduits 19 which connect the high-pressure gas source 18 to the atomizer 10. More than one atomizer 10 can be fixed on the rotatable telescopic support 12 depending on the size of drum coater 2 and the filling level of drug cores.

The ventilation system comprises an air inlet duct 7, a trapezoidal duct 9, an air outlet duct 4, a filter 5 and a fan 21. One side of the air inlet duct 7 is fixed at the top of housing 1 exposing to the air, and another side of the air inlet duct 7 connects to a trapezoidal duct 9 above the drum coater 2. The air outlet duct 4, the filter 5 and the fan 21 are installed below the drum coater 2, which are inside the housing 1. The fan 21 is fixed at the end of the filter 5. The fan 21 pumps air from the air inlet duct 7 towards the air outlet duct 4, which forms a negative pressure operation condition inside the operation box 3 and provides a good ventilation condition. The trapezoidal duct 9 and the air outlet duct 4 are installed on the opposite corners of the operation box 3, which are beneficial for maximizing the use of heat inside the operation box 3. To provide a better ventilation condition, more than one fans 21 can be installed depending on the size of operation box 3

The heating system comprises electric heating wires 8 installed inside the air inlet duct 7 and an electromagnetic heater 6 fixed below the drum coater 2. The electric heating wires 8 and electromagnetic heater 6 can work simultaneously or separately. The electromagnetic heater 6 is preferred if non-perforated drum coater is used for coating pellets, which increases the heat transfer rate and saves energy. During the coating process, the drug cores are loaded inside the drum coater 2 which is driven by a motor 20 and is rotating about its own axis. The drug cores are heated by the electric heating wires 8 or/and electromagnetic heater 6. If necessary, one or more heating rollers can be installed around the drum coater to increase the heating efficiency.

The ventilation system works throughout the coating process forming a negative pressure operation condition inside the operation box 3. The operation box 3 is well ventilated, hence greatly reducing the dust accumulation and the risk of dust explosion. The efficiency of powder coating can be improved a lot by selecting a proper nozzle for the corona spray gun 11 and vertically aiming the nozzle of the corona spray gun 11 and atomizer 10 at the rolling drug cores.

The auxiliary coating materials such as plasticizer in the liquid container 14 can be sprayed on the surface of drug cores through the atomizer 10 under the action of metering pump 15 and the high-pressure gas source 18. The use of plasticizer can reduce the surface resistance of the drug cores and enhance the adhesion rate of electrostatic powder to the surface of drug cores.

The corona spray gun 11 charges the dry powder and forms an electric potential difference between the charged powder and the drum coater 2. Affected by the electric field force, the charged dry powder adheres to the surface of drug cores. The spray zone of the atomizer is above the spray zone of the corona spray gun. The charged coating powers will immediately adhere to the surface of the drug cores after the liquid auxiliary coating materials are sprayed on the surface of the drug cores when the drug cores loaded inside the drum coater are rolling from up to down. As a result, both the utilization efficiency of auxiliary coating materials and the adhesion rate of charged powder can be greatly increased.

The electrostatic dry powder spraying and plasticizer spraying can be performed simultaneously or separately. As the drum coater 2 keeps on rotating, the auxiliary coating materials and the dry powder uniformly adhere to the surface of drug cores. By heating the drug cores to a suitable temperature such as the glass transition temperature of the polymers contained in the dry powder, a film is rapidly formed on the surface drug cores.

The touch screen control panel 24 can display the operating parameters of the dry powder coating apparatus, such as temperature, humidity, rotation speed of drum coater 2, amount of dry powder sprayed by the corona spray gun 11, flow rate of atomizer 10, location of rotatable telescopic support 12, system errors, etc. Above parameters are adjustable by touching the buttons on touch screen control panel 24. To form a better coating film and satisfy the needs of different gaining weights, the spraying of electrostatic powder and plasticizer may be performed several times.

In summary, the present disclosure provides an embodiment of a dry powder coating apparatus for coating pharmaceutical solid dosage forms, comprising:

a drum coater having a hollow interior, the drum coater being mounted inside a housing and being rotatable about its longitudinal axis;

a powder coating system for spraying a dry powder inside the hollow interior of the drum coater;

a liquid spray system for spraying a liquid containing auxiliary materials inside the hollow interior of the drum coater;

a heating system for heating the hollow interior of the drum coater, the heating system being positioned adjacent to the drum coater;

an operation box that encloses the drum coater in an operation volume and the operation box separates the operation volume from an external environment;

a ventilation system for air circulation between the operation volume and the external environment, wherein the ventilation system has an air inlet duct and an air outlet duct; and

a touch screen control panel attached to the housing and the touch screen control panel is configured to display at least one operating parameter of the dry powder coating apparatus.

In an embodiment the drum coater of the dry powder coating apparatus has a truncated cone on both sides, in which one side is opened and another side is closed.

In an embodiment the drum coater is horizontally placed inside the operation box along its axis and fixed on a rotatable shaft.

In an embodiment a motor is used to drive the shaft of the drum coater and rotates the drum coater about its longitudinal axis.

In an embodiment the drum coater is horizontally placed inside the operation box along its longitudinal axis and fixed on a rotatable shaft.

In an embodiment a motor is mechanically connected to the drum coater such that the motor can rotate the drum coater about the longitudinal axis.

In an embodiment the wall of drum coater is non-perforated, fully perforated or partially perforated. In an embodiment, each hole size in the fully or partially perforated wall may be around 1-10 mm, depending on the size of tablets to be coated.

In an embodiment the dry powder coating apparatus further comprises at least two or more baffles installed inside the wall of the drum coater. The baffles and the inner wall of the drum coater may be fully or partially coated with or made of insulating materials.

In an embodiment, the powder coating system is for forming a charged dry powder spray zone inside the drum coater, which comprises at least one corona spray gun which is fixed on at least one rotatable telescopic support, a powder container, powder delivery conduits which connect the powder container and a high-pressure gas source to the corona spray gun.

In an embodiment, the liquid spray system is for forming a liquid spray zone inside the drum coater, which comprises at least one atomizer which is fixed on at least one rotatable telescopic support, a metering pump, a liquid container, liquid delivery conduits which connect the atomizer with the metering pump and the liquid container, and gas delivery conduits which connect the atomizer with a high-pressure gas source.

In an embodiment the corona spray gun has a nozzle having a variety of 0-90° spray angle options for controlling the direction and the shape of the corona spray gun's spray zone. In an embodiment the corona spray gun shares a rotatable telescopic support with the atomizer.

In an embodiment the liquid spray zone is above the charged dry powder spray zone.

In an embodiment the one side of the rotatable telescopic support is fixed on the housing, and another side may be inserted into the drum coater and adjusted above drug cores by touching the control buttons on the touch screen control panel.

In an embodiment the ventilation system includes a trapezoidal duct above the drum coater.

In an embodiment the heating system includes heating wires inside the air inlet duct and an electromagnetic heater which is installed below the drum coater.

In an embodiment the drum coater is heated by a power adjustable electromagnetic heater installed below the coater or by the electric heating wires inside the air inlet duct, or simultaneously heated by both power adjustable electric heating wires and electromagnetic heater.

In an embodiment the touch screen control panel may be fixed on the housing. The touch screen control panel displays the operation conditions of the drum coater selected from the group consisting of temperature, humidity, rotation speed, the amount of powder sprayed, flow rate, location of rotatable telescopic support, and systems errors.

In an embodiment the operation box is inside the housing.

In an embodiment the drum coater has a cylindrical shape.

In an embodiment the air outlet duct has a filter therein and being connected to a fan such that the ventilation system is configured to generate negative air pressure in the operation volume.

The foregoing detailed description of the embodiment of the present disclosure is intended to illustrate the principles of the disclosure and not to limit the disclosure to the embodiment illustrated. Drawings are not drawn to scale. Detailed descriptions of well-known or conventional concepts are not described to provide a brief discussion of embodiment of the present disclosure. Technicians skilled in the art should understand that any modifications, equivalent substitutions, modifications, etc. to the present disclosure are intended to be included within the scope of the present disclosure.

Various embodiments and aspects of the disclosure are described in the detailed description. The description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components. It is intended that the scope of the disclosure is defined by all the embodiments encompassed within the following claims and their equivalents.

Therefore what is claimed is:

1. A dry powder coating apparatus for coating pharmaceutical solid dosage forms, comprising:
a drum coater having a hollow interior, the drum coater being mounted inside a housing and being rotatable about its longitudinal axis;
a powder coating system for spraying a dry powder inside the hollow interior of the drum coater;
a liquid spray system for spraying a liquid containing auxiliary materials inside the hollow interior of the drum coater;
a heating system for heating the hollow interior of the drum coater, the heating system being positioned adjacent to the drum coater;
an operation box that encloses the drum coater in an operation volume and the operation box separates the operation volume from an external environment;
a ventilation system for air circulation between the operation volume and the external environment, wherein the ventilation system has an air inlet duct and an air outlet duct; and
a touch screen control panel attached to the housing and the touch screen control panel is configured to display at least one operating parameter of the dry powder coating apparatus.

2. The dry powder coating apparatus of claim 1, wherein the drum coater has a truncated cone on both sides, in which one side is opened and another side is closed.

3. The dry powder coating apparatus of claim 1, wherein the drum coater is horizontally placed inside the operation box along its longitudinal axis and fixed on a rotatable shaft.

4. The dry powder coating apparatus of claim 1, wherein a motor is mechanically connected to the drum coater such that the motor can rotate the drum coater about the longitudinal axis.

5. The dry powder coating apparatus of claim 1, wherein the wall of drum coater is non-perforated, fully perforated or partially perforated, and wherein each hole size in the fully or partially perforated wall is around 1-10 mm, depending on the size of tablets to be coated.

6. The dry powder coating apparatus of claim 1, further comprising at least two or more baffles installed inside the wall of the drum coater.

7. The dry powder coating apparatus of claim 6, wherein the baffles and the inner wall of the drum coater are fully or partially coated with or made of insulating materials.

8. The dry powder coating apparatus of claim 1, wherein the powder coating system is for forming a charged dry powder spray zone inside the drum coater, comprising at least one corona spray gun which is fixed on at least one rotatable telescopic support, a powder container, powder delivery conduits which connect the powder container and a high-pressure gas source to the corona spray gun.

9. The dry powder coating apparatus of claim 1, wherein the liquid spray system is for forming a liquid spray zone inside the drum coater, comprising at least one atomizer which is fixed on at least one rotatable telescopic support, a metering pump, a liquid container, liquid delivery conduits which connect the atomizer with the metering pump and the liquid container, and gas delivery conduits which connect the atomizer with a high-pressure gas source.

10. The dry powder coating apparatus of claim 8, wherein the corona spray gun has a nozzle having a variety of 0-90° spray angle options for controlling the direction and the shape of the corona spray gun's spray zone.

11. The dry powder coating apparatus of claim 8, wherein the liquid spray zone is above the charged dry powder spray zone.

12. The dry powder coating apparatus of claim 8, wherein the corona spray gun shares a rotatable telescopic support with the atomizer.

13. The dry powder coating apparatus of claim 12, wherein one side of the rotatable telescopic support is fixed to the housing, and another side is inserted into the drum coater and adjusted above drug cores by touching the control buttons on the touch screen control panel.

14. The dry powder coating apparatus of claim 1, wherein the ventilation system includes a trapezoidal duct above the drum coater.

15. The dry powder coating apparatus of claim 1, wherein the heating system includes heating wires inside the air inlet duct and an electromagnetic heater which is installed below the drum coater.

16. The dry powder coating apparatus of claim 1, wherein the drum coater is heated by a power adjustable electromagnetic heater installed below the coater or by the electric heating wires inside the air inlet duct, or simultaneously heated by both power adjustable electric heating wires and electromagnetic heater.

17. The dry powder coating apparatus of claim 1, wherein the at least on operating parameter at least one of temperature, humidity, rotation speed, the amount of powder sprayed, flow rate, location of rotatable telescopic support, and systems errors.

18. The dry powder coating apparatus of claim 1, wherein the operation box is inside the housing.

19. The dry powder coating apparatus of claim 1, wherein the drum coater is generally cylindrical in shape.

20. The dry powder coating apparatus of claim 1, wherein the air outlet duct has a filter therein and being connected to a fan such that the ventilation system is configured to generate negative air pressure in the operation volume.
